(19) United States
(12) Patent Application Publication
(10) Pub. No.: US 2026/0364056 A1
(43) Pub. Date: Nov. 19, 2026

(54) METHODS OF TREATMENT OF NON-PANCREATIC GASTROINTESTINAL CANCERS

(71) Applicant: IONCTURA bv, Amsterdam (NL)

(72) Inventors: Gianluigi GIANNELLI, Castellana Grotte (IT); Giusy DI CONZA, Geneva (CH)

(21) Appl. No.: 19/488,226

(22) PCT Filed: May 31, 2024
(86) PCT No.: PCT/EP2024/064995

§ 371 Date: Nov. 26, 2025

(30) Foreign Application Priority Data
May 31, 2023 (GB) 2308140.9

(51) Int. Cl. A61K 31/437; A61K 31/136; A61K 31/282; A61K 31/4745; A61K 31/513; A61K 31/7048; A61K 31/7068; A61K 33/243; A61P 35/00

(52) U.S. Cl. CPC A61K 31/437; A61K 31/136; A61K 31/282; A61K 31/4745; A61K 31/513; A61K 31/7048; A61K 31/7068; A61K 33/243; A61P 35/00

(57) ABSTRACT

A compound of Formula (I) or a pharmaceutically acceptable salt thereof, for use in a method of treatment of a non-pancreatic gastrointestinal cancer.

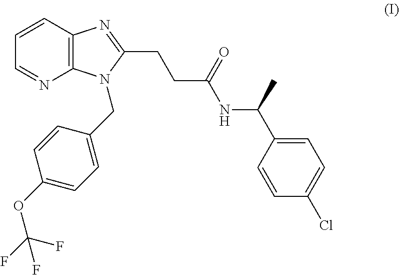 [SMILES: C[C@H](NC(=O)CCc1nc2cccnc2n1Cc1ccc(OC(F)(F)F)cc1)c1ccc(Cl)cc1]

### FIELD OF THE INVENTION

The present invention relates to a compound or pharmaceutically acceptable salt thereof for use in a method of treatment of non-pancreatic gastrointestinal cancers.

### BACKGROUND

Gastrointestinal cancer is cancer that develops along the GI tract (also known as the digestive tract). The GI tract starts at the oesophagus and ends at the anus. Primary gastrointestinal cancer starts growing in the GI tract. There were an estimated 4.8 million new cases of gastrointestinal (GI) cancers and 3.4 million related deaths, worldwide, in 2018. GI cancers account for 26% of the global cancer incidence and 35% of all cancer-related deaths (Arnold et al Gastroenterology 159(1) July 2020). The principle malignant conditions of the GI tract, namely cancers of the stomach (approximately 1.0 million new cases in 2018), liver (840 000 cases), oesophagus (570 000 cases), pancreas (460 000 cases), and colorectum (1.8 million cases), share a few common risk factors, but are largely distinct in their etiologies and descriptive epidemiologic profiles.

Based on projected changes in the age composition and growth of the world population, the global number of new cases of, and deaths from GI cancers are predicted to increase by 58% and 73% to 7.5 and 5.6 million, respectively, by 2040. The extent of the pending burden serves to both highlight the necessity of future clinical services planning for GI cancers, and the need to prioritize new targeted therapy in gastrointestinal cancer treatment. Targeted therapy is directed to specific receptors or enzymes that are present in the tumour and does not harm the healthy tissue unlike the traditional therapeutic methods like chemotherapy.

In 2018, colorectal cancer remains the most commonly diagnosed GI cancer, representing 1.8 million cases and 881 000 deaths globally, and constituting one in ten cancer cases and deaths. New diagnoses of colorectal cancers are surging in young adults and the disease is now the leading cause of cancer death in people 20 to 45 years old.

Colorectal cancers can be broadly subdivided into colon, rectal and anal cancers; while colon and rectal cancer share a number of risk factors, a large proportion of anal cancers have been attributed to infection with human papilloma virus (HPV), most notably HPV-16.

Liver cancer was the sixth most commonly diagnosed cancer (841,000 cases) and the fourth leading cause of cancer death (782,000 deaths) globally. Primary liver cancer can be broadly subdivided into hepatocellular carcinoma (HCC) (typically representing 75-85% of all liver cancer cases) and intrahepatic cholangiocarcinoma (ICC) (representing about 10-15% of cases), and other, more rare types.

Cholangiocarcinoma is bile duct cancer, and most cases of bile duct cancer cannot be cured. If the cancer is diagnosed at an early stage the 5-year survival rate is approximately 24%. However, if the cancer has spread to the regional lymph nodes, the 5-year survival rate reduces to 9%. If the cancer has spread to a distant part of the body, the 5-year survival rate is only 2%. Therefore, new therapies to improve survival rates are needed.

Lysophosphatidic acid (LPA) is a bioactive phospholipid that can act as a signalling molecule, regulating many cellular effects in various cell types. LPA can be synthesized by an intracellular pathway, involving phospholipids or diacylglicerol as precursors, and an extracellular pathway in which LPA is generated from lysophosphatidylcholine, normally present in the extracellular leaflet of plasma membranes or bound to other proteins, such as albumin.

Lysophosphatidylcholine is converted to LPA by Autotaxin (ATX), a secreted glycoprotein with lysophospholipase D activity encoded by the gene ectonucleotide pyrophosphatase phosphodiesterase 2 (ENPP2).

There are five ATX isoforms distributed in different tissues. This enzyme is composed of two N-terminal somatomedin B (SMB)-like domains, a central phosphodiesterase (PDE) domain containing the active catalytic site, and a C-terminal nuclease (NUC)-like domain. The N-terminal SMB-like domains are enriched with cysteine and are involved in the interaction of ATX with β3 integrins on the cell surface. The PDE domain also contains a hydrophobic lipid binding pocket that can bind different LPC and LPA species. The binding of ATX with integrins localize ATX activity to the cell surface, allowing the generation and delivery of LPA in the vicinity of its surface receptors. LPA interacts with six different G protein-coupled receptors (LPAR 1-6) to activate several pathways and downstream signalling molecules such as Rho, Ras PLC, PI3K, regulating physiological processes such as cell survival, proliferation and motility.

It is well known that ATX and its product LPA play an important role in physiological and pathological conditions. In healthy individuals, ATX is normally expressed in various tissue and can be found in biological fluids, but its expression has been found to be increased in several tumor types. In patients with hepatocellular carcinoma (HCC) it has been observed an overexpression of ATX respect to hepatitis C and healthy patients, and increased levels of ATX and LPA have been observed in lung tissue from patients with idiopathic pulmonary fibrosis and lung cancer. ATX gene is strongly up-regulated in kidney tumour tissue samples from RCC patients.

Given the prominent role of the ATX/LPA axis in promoting carcinogenesis, it represents a promising target for the treatment of cancer and inflammation-related diseases, such as chronic hepatitis and pulmonary fibrosis.

Several small molecule ATX inhibitors have been developed in recent years. In published patent application WO2016/124939, a series of novel ATX inhibitors and their use in treating medical conditions like cancer, inflammation, pain, diabetes mellitus, hypertension, atherosclerosis, thrombosis, urethral obstructive disease, fibrosis, hepatitis B and C and/or pruritus has been described. One particular compound disclosed in WO2016/124939 is a compound of Formula I.

This compound inhibits ATX with high selectivity and has shown remarkable effects in relevant disease models including cancers containing fibrosis. For example, in a mouse model for lung fibrosis, the compound showed a greater reduction in fibrosis score compared with GLPG1690 and comparable activity with gemcitabine in a mouse model for pancreatic cancer. Thus, a compound of Formula I has great potential for use in human patients.

There is increasing demand for targeted therapy in gastrointestinal cancer treatment. Targeted therapy is directed to specific receptors or enzymes that are present in the tumour and does not harm the healthy tissue unlike the traditional therapeutic methods like chemotherapy. The present invention has been devised in light of the above considerations.

### SUMMARY OF THE INVENTION

The present invention is directed to a compound for use in the treatment of non-pancreatic gastrointestinal cancers. The compound is an ATX inhibitor. The inventors recognised that ATX inhibitors may be useful in targeted therapy for the treatment of non-pancreatic gastrointestinal cancers, such as liver cancer, cholangiocarcinoma or colorectal cancer.

In a first aspect, the invention provides a compound of Formula I:

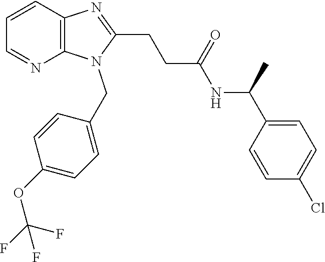 [SMILES: C[C@H](NC(=O)CCc1nc2cccnc2n1Cc1ccc(OC(F)(F)F)cc1)c1ccc(Cl)cc1]

or a pharmaceutically acceptable salt thereof for use in the treatment of non-pancreatic gastrointestinal cancers in a patient. The compound of Formula I may be referred to herein as “Compound 1”.

The term non-pancreatic gastrointestinal cancer includes anal cancer, bile duct cancer, colon cancer, oesophageal cancer, gallbladder cancer, gastrointestinal stromal tumours, liver cancer rectal cancer, colorectal cancer, small intestine cancer, stomach (gastric) cancer and cholangiocarcinoma, stomach cancer includes adenocarcinomas, gastrointestinal stromal tumours and neuroendocrine tumours (including carcinoids).

In some embodiments the invention provides Compound 1 or a pharmaceutically acceptable salt thereof for use in the treatment of liver cancer, cholangiocarcinoma or colorectal cancer.

In another embodiment the invention provides a method of treatment of liver cancer, cholangiocarcinoma or colorectal cancer comprising administering Compound 1 to a patient.

In another embodiment the present invention provides the use of Compound 1 for the manufacture of a medicament for the treatment of liver cancer, cholangiocarcinoma or colorectal cancer.

Suitably, Compound 1 is administered in a pharmaceutical composition comprising Compound 1 or a pharmaceutically acceptable salt thereof and a pharmaceutically acceptable diluent, carrier or excipient. Preferably, but not necessarily, the pharmaceutical composition is suitable for oral administration.

In some cases, the treatment is a combination therapy and comprises one or more additional chemotherapeutic agents. For example, the method may comprise administration of a therapeutically effective amount of an additional chemotherapeutic agent, optionally two additional chemotherapeutic agents. In these embodiments, disclosed is Compound 1, or a pharmaceutically acceptable salt thereof, for use in the treatment of a non-pancreatic gastrointestinal cancer in a patient, wherein said treatment comprises the separate, sequential or simultaneous administration of i) said Compound 1, or a pharmaceutically acceptable salt thereof, and ii) one or more additional chemotherapeutic agents, or pharmaceutically acceptable salts thereof, to said patient. In further embodiments disclosed is the use of Compound 1, or a pharmaceutically acceptable salt thereof, in the manufacture of a medicament for use in the treatment of a non-pancreatic gastrointestinal cancer, wherein said treatment comprises the separate, sequential or simultaneous administration of i) said medicament comprising Compound 1, or a pharmaceutically acceptable salt thereof, and ii) one or more additional chemotherapeutic agents, or pharmaceutically acceptable salts thereof, to said patient. In further embodiments disclosed is a method of treating a non-pancreatic gastrointestinal cancer in a patient in need thereof, comprising administering to the subject a first amount of Compound 1 or a pharmaceutically acceptable salt thereof, and a second amount of one or more additional chemotherapeutic agents, or pharmaceutically acceptable salts thereof. In the method, the first amount and the second amount together comprise a therapeutically effective amount.

In the fight against gastrointestinal tumors, the strategy of using drugs that target the signalling pathway represented by the ATX/LPA axis can represent a novel therapeutic option in association with common chemotherapy drugs, in particular, in the treatment of those tumors characterized by a high level of fibrosis, such as but not limited to liver cancer. The examples demonstrate that, in this context, Compound 1 plays an important role.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1A shows density of cells incubated with Compound 1 at the indicated concentrations for 72 h in culture medium supplemented with 10% FBS. FIG. 1B shows different cell response to treatment with 9 μM Compound 1 for 72 h in the absence of and in the presence of 2 and 5% FBS.

FIGS. 2A and 2B show the effect of Compound 1 on cell proliferation across multiple cell lines in the presence of 10% FBS and in the absence of FBS.

FIGS. 3A and 3B shows the effect of Compound 1 on cell migration. FIG. 3A shows representative phase-contrast microscope images and quantification of a wound healing assay of cells treated with Compound 1 in serum-free medium, for 48 hours. FIG. 3B shows representative microscopic images and quantification of transwell migration assay of cells treated with 12 μM Compound 1 for 24 hours, showing a decrease in the number of migrated cells through the membrane.

FIG. 4 shows fluorescence microscopy of cells treated with 3, 9 and 12 μM Compound 1 for 4 hours and stained with Annexin V-FITC antibody.

FIG. 5A shows representative pictures and quantification of the differentially expressed apoptosis-related proteins between untreated control cells and cells incubated with 12 μM Compound 1 for 24 h.

FIG. 5B shows a list of proteins detected by the human apoptosis antibody array.

FIG. 6 shows crystal violet staining (top) and morphological changes (bottom) of KKU-M213 cells observed under an inverted light microscope following treatment with 3, 9 and 12 μM Compound 1 for 72 hours.

FIG. 7 shows bright-field images and cell proliferation assay measurements of isolated intrahepatic cholangiocarcinoma cancer cells treated with Compound 1 at 3, 9 and 12 μm.

FIG. 8 shows the effect on KKUM-213 xenografted tumour volume following treatment by Compound 1.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Compound 1

WO2016/124939 describes various ATX inhibitor compounds and their use in the treatment of proliferative disorders in which ATX activity is implicated, including Compound 1.

Compound 1 is example 40 in WO2016/124939, which document is incorporated herein by reference in its entirety. WO2016/124939 describes over 200 examples. Compound 1's structure is according to Formula I.

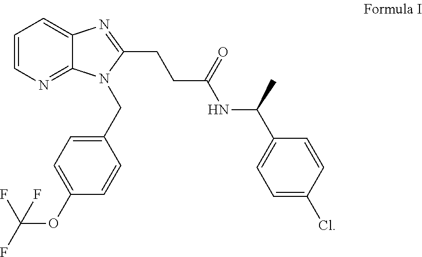 [SMILES: C[C@H](NC(=O)CCc1nc2cccnc2n1Cc1ccc(OC(F)(F)F)cc1)c1ccc(Cl)cc1]

Its IUPAC name is N-[(S)-1-(4-chloro-phenyl)-ethyl]-3-[3-(4-trifluoromethoxy-benzyl)-3H-imidazo[4,5-b]pyridin-2-yl]-propionamide. Its synthesis and characterisation are described in WO2016/124939 at pages 77 and 82, respectively, which information is specifically incorporated herein by reference.

Compound 1 may be provided and administered as the free base or as a pharmaceutically acceptable salt. In some cases, Compound 1 is provided and administered as the free base.

### Formulation

Suitability, Compound 1 is provided in a pharmaceutical composition formulated for oral administration. The pharmaceutical composition may be provided in a capsule or may be provided in a tablet. In some cases, it is provided in a tablet. In other cases, it is provided in a capsule, for example, as a powdered or granulated composition or a liquid composition within a hard- or soft-shell capsule, for example, a hydroxymethyl cellulose (HPMC) capsule. In other words, an oral dosage form is preferred.

The formulation suitably comprises one or more pharmaceutically acceptable fillers, Disintegrants, glidants, and/or lubricants.

### Additional Chemotherapeutic Agent

The one or more additional chemotherapeutic agents may be selected from those which are currently the standard of care for treating the particular non-pancreatic gastrointestinal cancers.

In some embodiments, the one or more additional chemotherapeutic agents may be selected from gemcitabine, cisplatin, doxorubicin, mitoxantrone, 5-fluorouracil (5-FU), capecitabine, irinotecan, oxaliplatin, trifluridine and tipiracil.

### Treatment of Non-Pancreatic Gastrointestinal Cancers Using Compound 1

Compound 1 is an ATX inhibitor. ATX levels directly correlate with stage and grade in several human cancers. Its product, LPA, stimulates cell proliferation and migration and promotes wound repair following tissue damage.

It is well known that ATX and its product LPA play an important role in physiological and pathological conditions. In healthy individuals, ATX is normally expressed in various tissue and can be found in biological fluids but its expression has been found to be increased in several tumor types including non-pancreatic gastrointestinal cancers. The methods of the present invention may therefore relate to treatment of non-pancreatic gastrointestinal cancers characterised by high ATX expression. The methods of the present invention may therefore relate to treatment of non-pancreatic gastrointestinal cancers by modulation of ATX-LPA pathway in a patient.

Compound 1 does not bind to the catalytic zinc region of ATX but binds to both the substrate pocket and the LPA carrier channel blocking both functions of ATX. Compound 1 selectively inhibits LPA, leading to an anti-proliferative effect in various gastrointestinal cancers.

The gastrointestinal cancer may be liver cancer, cholangiocarcinoma or colorectal cancer. Accordingly, the methods of the present invention are directed towards the treatment of gastrointestinal cancers, such as but not limited to liver cancer, cholangiocarcinoma, or colorectal cancer. In some cases, the gastrointestinal cancer is liver cancer. In some cases, the gastrointestinal cancer is cholangiocarcinoma. In some cases, the gastrointestinal cancer is colorectal cancer.

### METHODS OF THE INVENTION

As described in more detail below, the present inventors have surprisingly found that Compound 1 shows robust anti-tumour activity in preclinical models of various gastrointestinal cancers and is well tolerated. Thus, an ATX inhibitor with anti-tumour activity and favourable safety characteristics in gastrointestinal cancers selected from liver cancer, cholangiocarcinoma and colorectal cancer can be provided.

The term non-pancreatic gastrointestinal cancer can include for example anal cancer, bile duct cancer, colon cancer, oesophageal cancer, gallbladder cancer, gastrointestinal stromal tumours, liver cancer, rectal cancer, colorectal cancer, small intestine cancer, stomach (gastric) cancer and cholangiocarcinoma. Stomach cancer includes adenocarcinomas, gastrointestinal stromal tumours and neuroendocrine tumours (including carcinoids). Other types of cancer such as squamous cell carcinomas, small cell carcinomas and leiomyosarconmas, can also start in the stomach.

In particular, Compound 1 shows robust anti-tumour activity in preclinical models of liver cancer, cholangiocarcinoma and colorectal cancer. Thus, provided is Compound 1 for use in a method of treatment of liver cancer, cholangiocarcinoma or colorectal cancer.

Colorectal cancer starts in the colon or the rectum. These cancers can also be called colon or rectal cancer depending on where they start. Several types of colon cancer exist including adenocarcinoma, carcinoids, familial colorectal cancer (FCC), Gastrointestinal stromal tumours (GIST), squamous cell tumours, carcinoid tumours, sarcomas, rectal cancer, anal cancer, and cancer of the small bowel. More than 90% of colorectal carcinomas are adenocarcinomas originating from epithelial cells of the colorectal mucosa. Other rare types of colorectal carcinomas include primary colorectal lymphomas, gastrointestinal stromal tumours, colon and rectal leimyosarcomas, colon and rectal melanomas, colorectal squamous cell carcinoma, familial adenomatous polyposis (FAP), neuroendocrine, squamous cell, adenosquamous, spindle cell and undifferentiated carcinomas.

Liver cancer is also known as hepatic cancer, primary hepatic cancer or primary hepatic malignancy and is a cancer which starts in the liver. Liver cancer can be primary (starting in the liver) or secondary (starting elsewhere and spreading to the liver) known as liver metastasis. Types of liver cancer include Hepatocellular carcinoma (HCC), Fibromellar carcinoma, angiosarcoma, hepatoblastoma, bile duct cancer (cholangiocarcinoma) and metastasis to the liver.

Cholangiocarcinoma is a cancer that is found anywhere in the bile ducts and is a rare but aggressive form of cancer. The bile duct system is made up of a series of tubes that begin in the liver and end in the small intestine. There are two types of bile duct cancer intrahepatic bile duct cancer and extrahepatic bile duct cancer. Intrahepatic bile duct cancer forms in the bile ducts inside the liver. Only a small number of bile duct cancers are intrahepatic. Intrahepatic bile duct cancers are also called intrahepatic cholangiocarcinomas. Extrahepatic bile duct cancer forms in the bile ducts outside the liver and there are two types of extrahepatic bile duct cancer—perihilar bile duct cancer and distal bile duct cancer. Perihilar bile duct cancer is found in the area where the right and left bile duct exit the liver and join to form the common hepatic duct. Perihilar bile duct cancer is also called a Klatskin tumor or perihilar cholangiocarcinoma. Distal bile duct cancer is also called extrahepatic cholaniocarcinoma and is found in the area where the ducts from the liver and gallbladder join to form the common bile duct.

Therefore, the inventors have found that, surprisingly, various gastrointestinal cancers can be treated with Compound 1 with or without other chemotherapy and this treatment is well tolerated. This provides new monotherapy and combination therapy options for the treatment of various non-pancreatic gastrointestinal cancers, in particular liver cancer, cholangiocarcinoma and colorectal cancer.

The dose of Compound 1 may be provided once daily (QD), preferably twice daily (BID), preferably but not necessarily administered orally. Other methods of administration may be used. A suitable daily dose may be between 5 mg and 2 g, for example between 10 mg and 1 g. In some cases, where Compound 1 is administered in a combination therapy, administration of Compound 1 continues during pauses in administration of other agents (for example, during days 21-28 of 28 day chemotherapeutic cycles).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Materials and Methods

### Statistical Analysis

Tests were performed for statistical comparison between groups using GraphPad Prism 6.01 software (GraphPad Software Inc., La Jolla, CA, USA). Statistical significance was assumed when p<0.05.

### Example 1

### Materials and Methods

### Cell Culture and Reagents

Human colorectal adenocarcinoma HT29 and Caco-2 cells, hepatocellular carcinoma HLE cells, cholangiocarcinoma RBE and KKU-M213 cells were purchased from the American Tissue Culture Collection (ATCC, Manassas, VA, USA). The hepatocellular carcinoma cell line HLF was purchased from JCRB Cell Bank (Japan). All cell lines were cultured in Dulbecco's Modified Eagle Medium (DMEM) supplemented with 10% foetal bovine serum (FBS), 1 mM pyruvate, 25 mM HEPES, 100 U/mL penicillin-streptomycin (all from Thermo Fisher Scientific, Waltham, MA, USA), and maintained in a humidified atmosphere at 37 °C containing 5% CO₂. The medium was changed every three days. Oleoyl-LPA was purchased from Avanti Polar Lipids (Birmingham, AL).

### Cell Viability Assay

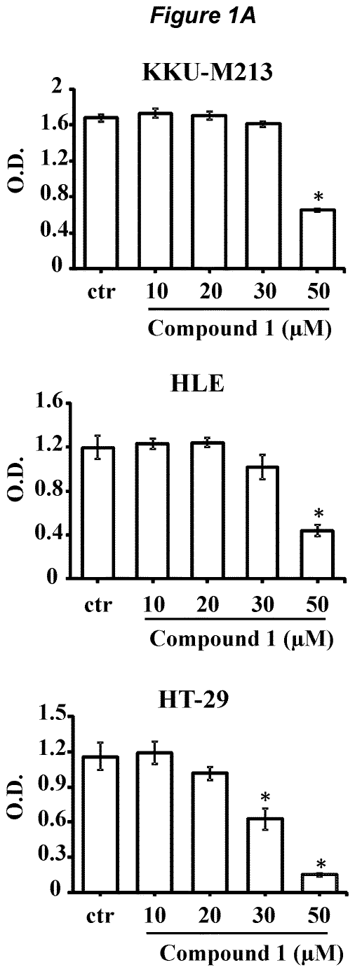

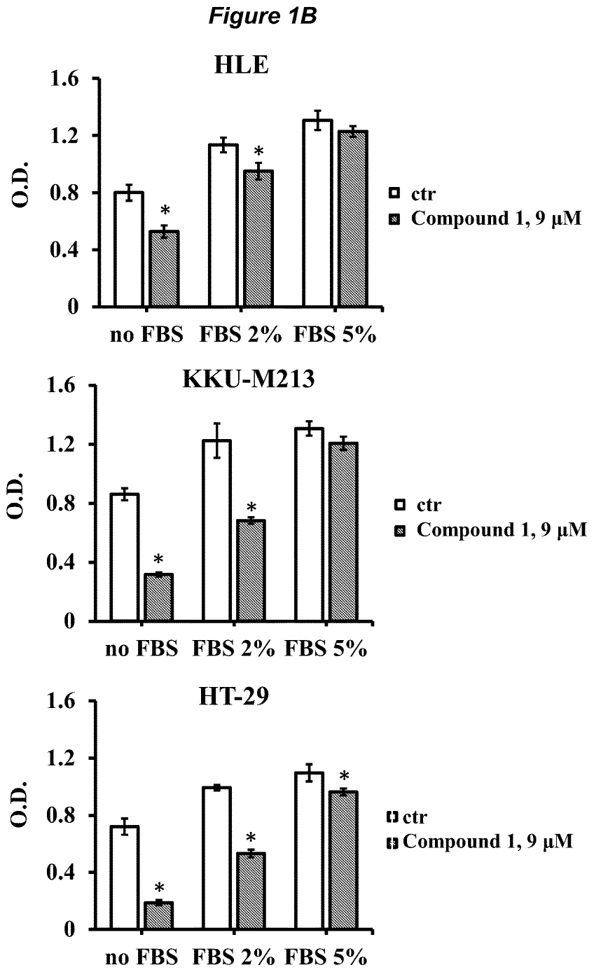

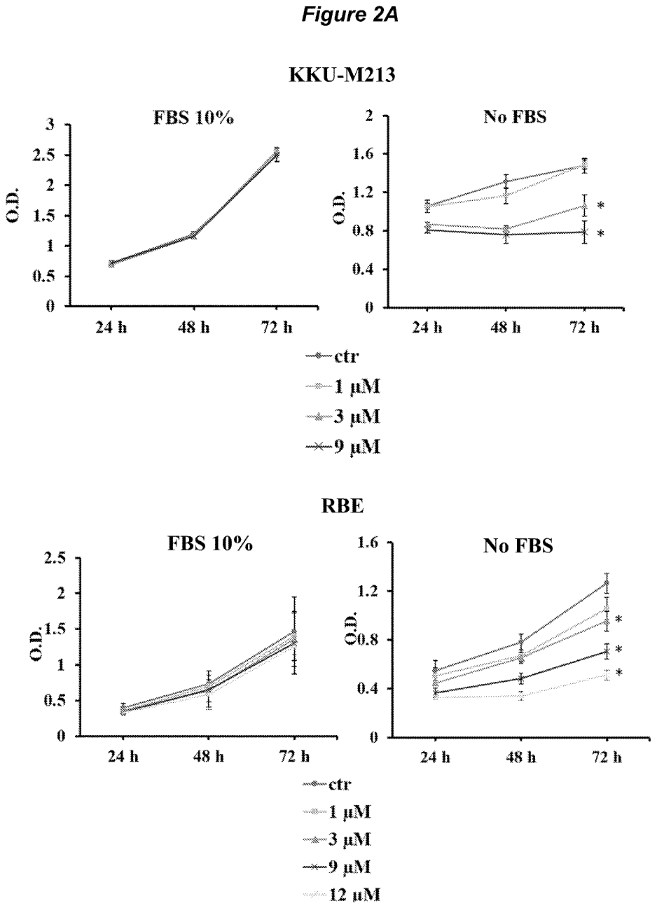

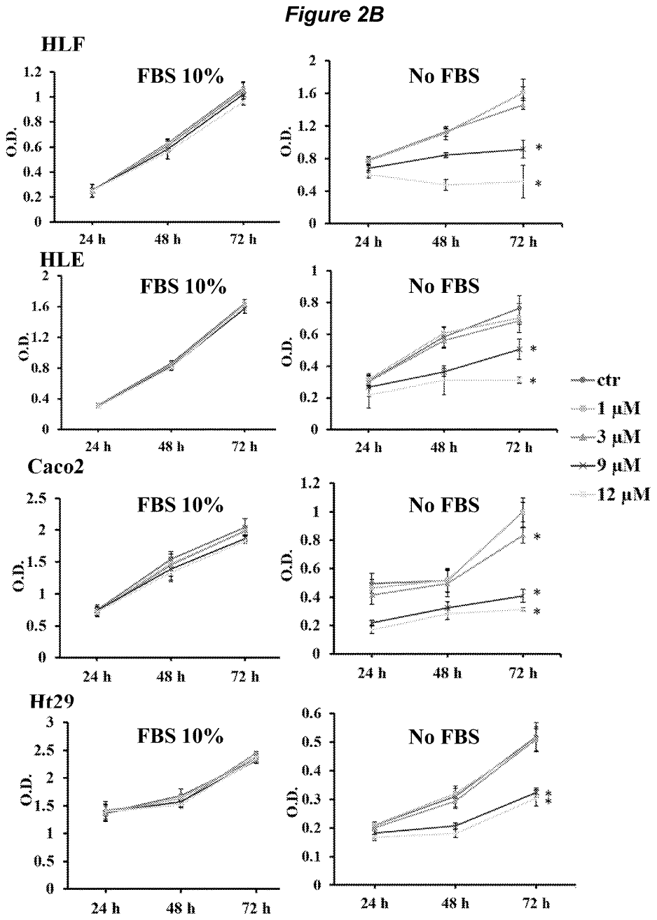

Cells were seeded in 96-well plates at a density of 2000 cells/well in 10% FBS supplemented medium. After 24 h, medium was removed, the cells washed with PBS and cultured in the presence or absence of FBS and of different concentrations of Compound land LPA. After the time set for the experiment, the cells were fixed with a 4% PFA solution and stained with 0.02% crystal violet for 10 min. Subsequently the plates have been washed with water to remove excess dye. The cell-bound dye was redissolved in 1% SDS and the optical density was measured at λ=570 with a iMark™ Microplate Absorbance Reader (Bio-rad). The results are shown in FIGS. 1A, 1B, 2A and 2B.

FIG. 1A shows the density of cells incubated with Compound 1 at the indicated concentrations for 72 h in culture medium supplemented with 10% FBS. FIG. 1A shows that Compound 1 does not modulate cell proliferation in presence of serum A comprising FBS.

FIG. 1B shows the different cell response to treatment with 9 μM Compound 1 for 72 h in the absence of and in the presence of 2 and 5% FBS.

FIGS. 1A and 1B together show that in the absence of foetal bovine serum (FBS), Compound 1 modulates cell proliferation whereas in the presence of FBS it does not. This is because in the presence of FBS the amount of lysophosphatidic acid (LPA) is amplified. The bioactive phospholipid lysophosphatidic acid stimulates cell proliferation, migration and survival by acting on its cognate G-protein-coupled receptors. LPA is a growth factor that also stimulates cell migration and can therefore contribute to both cancer progression and metastasis. LPA is produced by platelets, and it is known that there is high LPA in FBS. In the presence of LPA in the FBS the effect of the ATX inhibitor cannot be seen because the LPA interacts with the cells in the culture medium. Therefore, when the FBS is removed from the culture medium the effect of the ATX inhibitor can clearly be seen and it inhibits the cells on each of the tested cell lines. In FIG. 1B where there is no FBS there is a clear difference in the cell inhibition compared to the control in each of the HLE, KKU-M213 and HT-29 cell lines.

FIGS. 2A and 2B show the effect of Compound 1 on cell proliferation in the absence of FBS and in the presence of 10% FBS. FIGS. 2A and 2B demonstrate the inhibition of cell proliferation of the indicated gastrointestinal cancer cell lines by Compound 1. Cells were grown in serum free culture medium or in presence of 10% FBS and incubated with increasing Compound 1 concentrations or with DMSO (control cells) for 24, 48 and 72 hours. After fixation, proliferation was assessed by crystal violet staining method. *P<0.05.

FIGS. 2A and 2B also demonstrate the effect of the FBS on the inhibition of Compound 1 as outlined above. As shown in FIG. 2A, in the presence of FBS the effect of Compound 1 is generally the same as the control, whereas where there is no FBS the figure clearly shows clear inhibition in both the KKU-M213 and RBE cell lines. The same effect is shown in FIG. 2B in the HLF, HLE, Caco2 and HT-29 cell lines.

This shows that in the absence of FBS/LPA Compound 1 clearly inhibits the cancer cells across various gastrointestinal cell lines.

### Effects of Compound 1 on Cell Viability

To evaluate the anti-proliferative/cytotoxic effects of Compound 1, six different cancer tumour cell lines were treated developed from cholangiocarcinoma (CCA), hepatocellular carcinoma (HCC) and colorectal cancer (CRC), with increasing concentration of the Compound 1 ranging from 10 μM up to 50 μM and at different times of incubation. After 24, 48 and 72 hours of drug treatment, attached cells were fixed and stained with crystal violet dye. This method is used to evaluate the effect of chemotherapeutics or other anti-cancer drugs on cell survival and proliferation. In the first experiments, Compound 1 was administered to cells in culture medium supplemented with 10% FBS. Compound 1 showed a significant (p<0.05) cytotoxic effect at highest concentrations (30 to 50 μM, FIG. 1A) in all the cell lines used: KKU-M213, RBE, HLE, HLF, Caco2 and HT-29 cells. To test the hypothesis that the presence of serum, and consequently the endogenous LPA, could interfere with the Compound 1 activity, all the cancer cells were treated with Compound 1 at concentration ranging from 1 μM up to 12 μM, in the presence or absence of 10% FBS.

As reported in FIGS. 2A and 2B, Compound 1 displayed a significant (p<0.05) cytotoxic effect in a dose-dependent manner even at lower concentrations (3 to 12 μM). This effect, already evident after 48 hours, became stronger after 72 hours of incubation. Thus, Compound 1 shows cytotoxic effect in cells cultured in the absence of FBS but not in those where 10% FBS was present in the medium. These results were consistent for all the cancer models and were observed at each repetition (at least three independent experimental repetition). These results suggest that Compound 1 exerts a strong anti-proliferative effect in all the models and that FBS interferes with its activity. To further expand the preliminary observations, HLE, KKU-M213 and HT-29 cells were challenged with 9 μM of Compound 1 in the absence or presence of increasing concentration of FBS. In all the cell models, the presence of FBS significantly (p<0.05) reduced the activity of Compound 1 starting at 2% of FBS concentration and completely abolishing the Compound 1 effect at 5% FBS.

These results demonstrate that Compound 1 selectively inhibits LPA, leading to an anti-proliferative effect, however in presence of FBS the drug effectiveness is blocked likely to supraphysiological levels of endogenous LPA. LPA and ATX play a fundamental role in the progression of several tumors. Precisely because of this role, this axis can represent an important therapeutic target.

The biological effects of Compound 1 treatment have been characterised on different tumor cell lines of the gastrointestinal tract: cholangiocarcinoma (KKU-M213, RBE), hepatocellular carcinoma (HLE, HLF) and colorectal cancer (Caco2, HT-29). Consistently, Compound 1 hampered cell proliferation in all cell lines, in a dose-dependent manner and with stronger effect at later time points. Unexpectedly, the presence of FBS partially or completely inhibited the cytotoxic effects of Compound 1. This may be due to supraphysiological concentrations of LPA in FBS which consequently blunts the activity of Compound 1. In human foetal serum LPA is known to be highly active and hence it is reasonable to assume the FBS contains similar supraphysiolocial concentrations. Therefore, it is critical to be aware of the interference of FBS when assessing anti-tumor activity in cell cultures for novel ATX inhibitors.

The mortality associated with gastrointestinal tumors is mainly related to the predisposition to metastasize. For example, in animal models of breast cancer Compound 1 has shown to inhibit the dissemination of primary tumors. Hence, this shows that LPA inhibition is a key contributor to blocking metastasis. This is consistent with the role of LPA in stimulating tumor cell motility. For example, LPA stimulates the motility of DLD1 colon carcinoma cells and regulates the migration of gastric cancer cells. Through its receptor LPAR1, LPA induces the migration and expression of MMP9 in HCC cell lines. The LPA, ATX and LPA receptors are widely expressed in different tumor types and at different degrees. This differential expression observed in patients may also be present in cell lines and hence explain the occasional difference in the response to Compound 1 blocking the LPA signalling.

### Example 2

### Wound Healing Assay

Cells were seeded in a 12-well plate in 10% FBS medium and grown to reach a 80-90% confluence. Then, a scratch was made on the cell monolayer by using a p200 pipette tip. The plate was washed with sterile PBS to remove the debris and on the outer bottom of the plate were created markings with a tip marker, used as reference points. Each cell line was incubated with medium without FBS and containing increasing concentrations of Compound 1 (1-3-9-12 μM). Images were acquired at 0, 24 and 48 h by using a confocal microscope Nikon Eclipse Ti2 in bright field microscope and analysed with image-J. Image-J software was used to measure the scratched area. Cell migratory ability of wound-healing was assessed using the following formula: [(wound area at 0 h)−(wound area at indicated time)]/(wound area at 0 h).

### Transwell Migration Assay

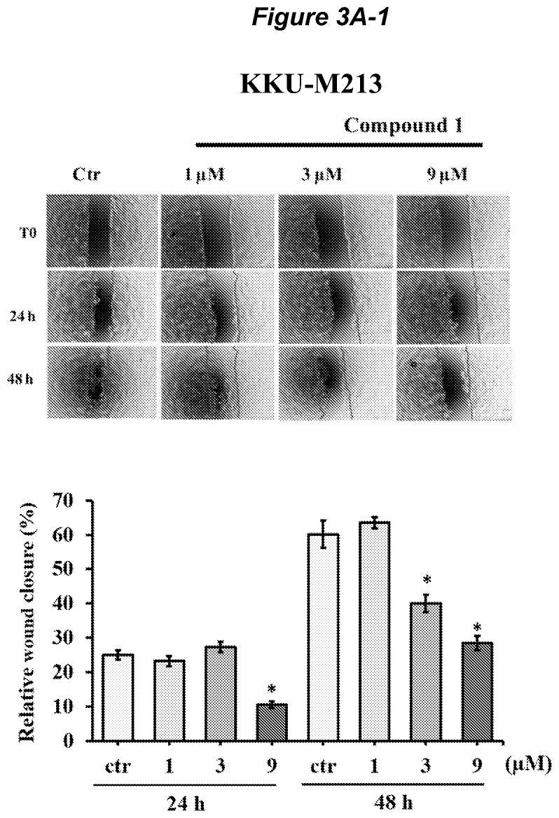

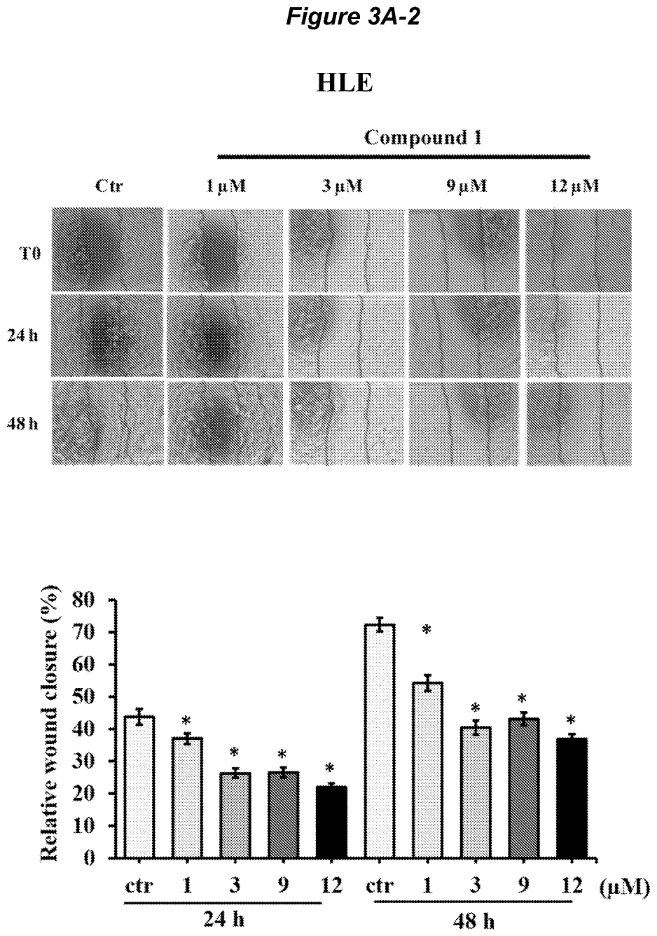

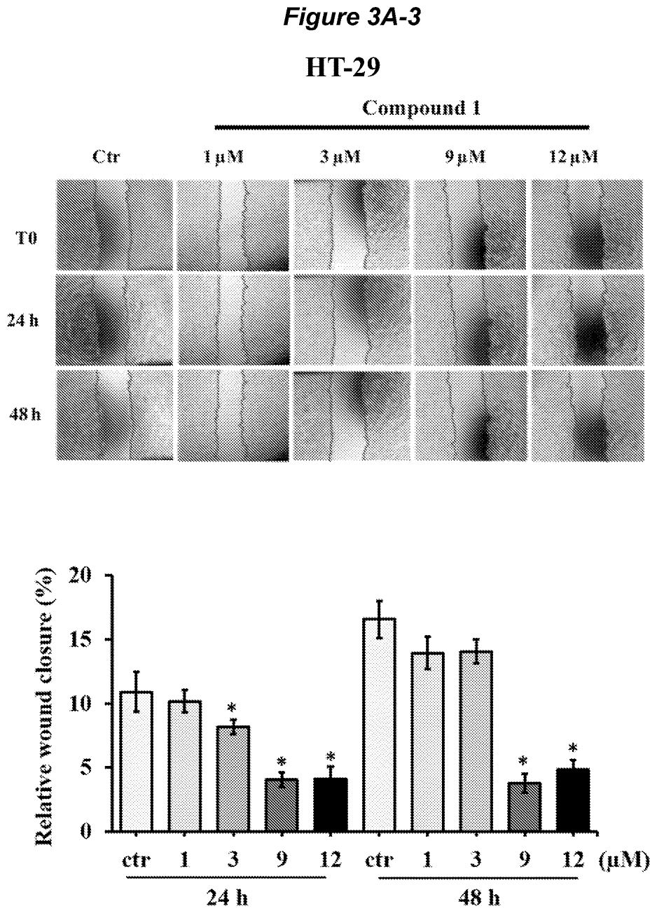

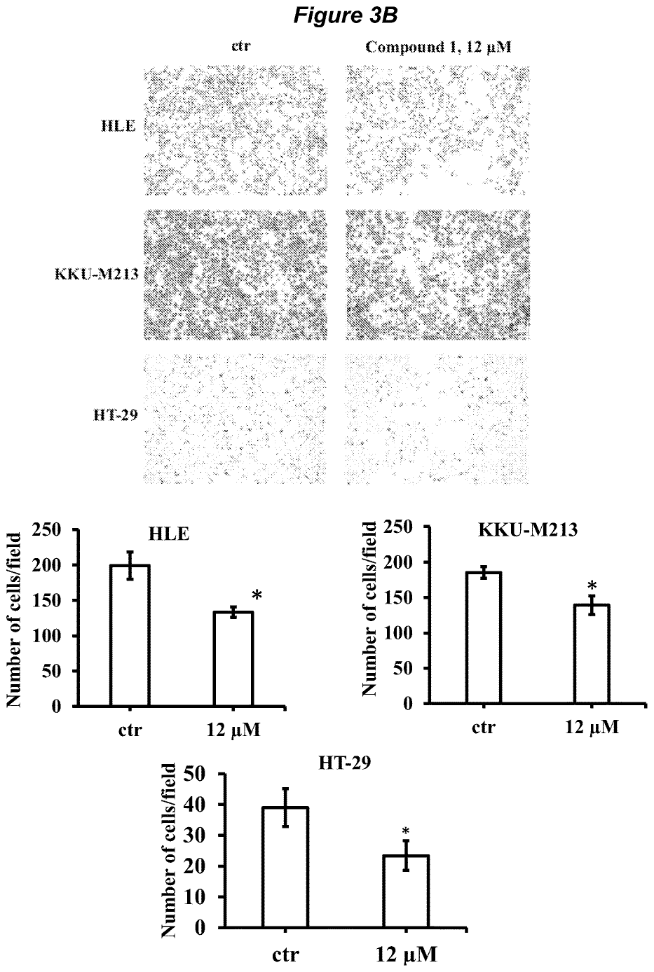

Cell culture inserts with 8 μM pores (Corning, inc.) were coated with 50 μl of a 10 mg/ml Collagen type 1 solution (Gibco). 1.5×10⁴ cells were plated in the upper chamber in serum-free medium, in absence or presence of 12 μM Compound 1, while the bottom wells were filled with complete medium. The cells were allowed to migrate across the membrane for at least 16 h. After incubation, the cells were fixed with PFA 4% for 10′ and stained with 0.02% crystal violet for 10′. The excess dye was washed off with tap water and the non-migrated cells were scraped off from the upper surface of the membrane with a cotton swab. Cell migratory ability was assessed by counting the average number of cells at least 5 field at 10× magnification. The results are shown in FIGS. 3A and 3B.

FIG. 3 shows the effect of Compound 1 on cell migration. FIG. 3A shows representative phase-contrast microscope images and quantification of a wound healing assay of cells treated with Compound 1 in serum-free medium, for 48 h. Images were taken at time 0, 24 h and 48 h and cell migration into the cell-free region (outlined) was quantified. FIG. 3B shows representative microscopic images and quantification of transwell migration assay of cells treated with 12 μM Compound 1 for 24 h, showing a decrease in the number of migrated cells through the membrane. *P<0.05

### Effect of Compound 1 on Cell Migration

Tumor progression is characterized by an enhanced cell motility and invasiveness. To expand upon the knowledge of the effects of Compound 1 on HLE, KKU-m213 and HT-29 cells, their migratory activity was assessed when treated with Compound 1 by using both, a wound healing and a transwell migration assay. Cells were seeded in a 12-well plate and, when a confluent monolayer was reached, an artificial gap was generated between the cells using a pipette tip. The scratch closure of migrating cells was observed after 48 h and 72 h. As reported in FIG. 3A, Compound 1 significantly (p<0.05) reduced cell migration and wound closure of KKUM-213 cells treated with 9 μM Compound 1 (approximately 50% of reduction in wound closure), respect to control, already after 24 h and 48 h of treatment. In HLE cells a 3 μM concentration of Compound 1 is already sufficient to cause a 40% inhibition of cell migration. In HT-29 cells 9 and 12 μM of Compound 1 significantly (p<0.05) inhibited wound closure. Similarly, the transwell assay showed that Compound 1 treatment significantly inhibited migratory capacity of all the tested cancer cell lines. (FIG. 3B). This demonstrates that Compound 1 inhibits cancer cell migration.

It has been observed, both by wound healing assay and transwell migration assay, that inhibition of ATX by Compound 1 decreases the motility of tumor cell lines, in a dose-dependent manner. Several studies have demonstrated that ATX and LPA can protect cells from apoptosis and confer resistance to chemotherapeutic drugs, as it has been observed, for example, in colon cancer cells. The examples demonstrate that Compound 1 treatment determines the activation of the apoptotic pathway, as shown by the exposure on the membrane of the phosphatidylserine residues detectable by staining with Annexin V-FITC and by the upregulation of several pro-apoptotic proteins such as BIM, Caspase-3 and -8, SMAC, Fas and HTRA, among others. Similar to observation in cancer cells, others reported LPA-dependent regulation in aortic smooth muscle cells. Again, the concentration of FBS were key in blocking the effect of LPA-associated cell death.

It has therefore been shown for the first time that ATX inhibitors have to be evaluated with care in conditions that do not obscure the potential effect under more physiological conditions. Since more patients have their tumors in a nutrient-depleted condition (REF), the observation in the absence of FBS are pointing to a potential direct tumor cell killing effect of ATX inhibition, which was until now underappreciated.

### Example 3

### Annexin V Staining

For detection of early apoptosis the Annexin V-FITC kit (Miltenyi Biotech) was used. Cells were seeded and grown on Nunc Lab-Tek II Chamber Slide. After a wash with PBS, cells were incubated with Compound 1 3, 9 and 12 μM in serum-free medium. After 2 hours cells were washed with Binding Buffer (BB) and stained with a solution of Annexin V-FITC antibody in BB for 15′. After 2 washes with BB the cells were fixed with a 4% paraformaldehyde solution and a coverslip was mounted onto the slide using a DAPI Ready Made Solution with Antifade (Merck). Images were acquired using a confocal microscope Nikon Eclipse Ti2 and analysed with image-J.

### Human Apoptosis Antibody Array

The Human Apoptosis Antibody Array Kit (ab134001, Abcam) was used to detect the apoptosis pathway. Briefly, cells were lysed and each membrane was incubated at 4 °C overnight with 500 μg of total extracted protein. Membranes were then washed and incubated overnight at 4 °C with Biotin-conjugated Anti-Cytokines. After an incubation of the membranes with HRP-streptavidin for 2 h at room temperature, chemiluminescence was detected using ChemiDoc XRS+ (Bio-Rad, Hercules, CA, USA). The images were analysed with Image Lab 5.2.1.

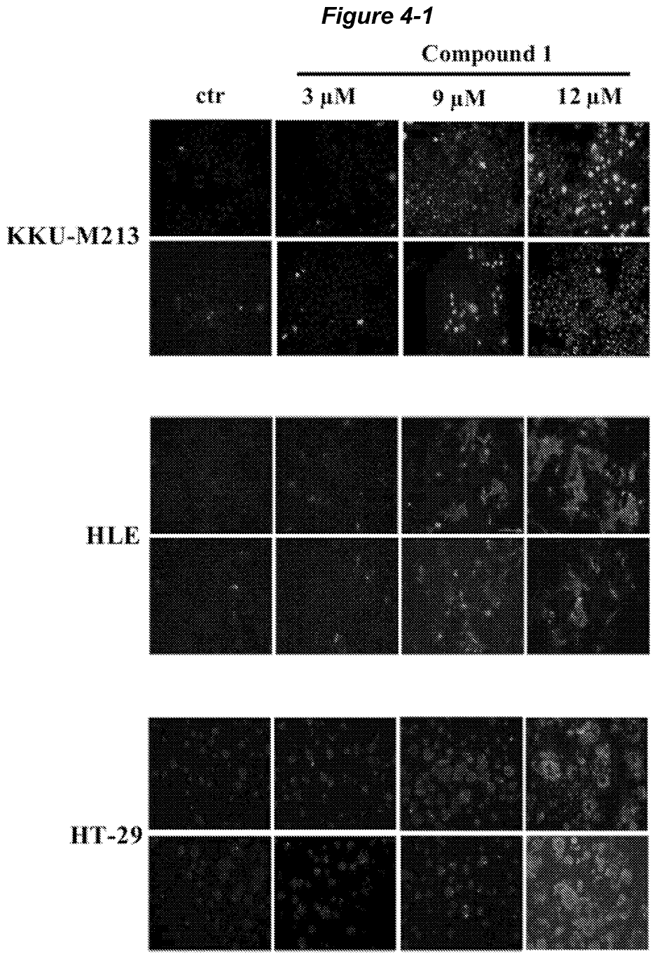

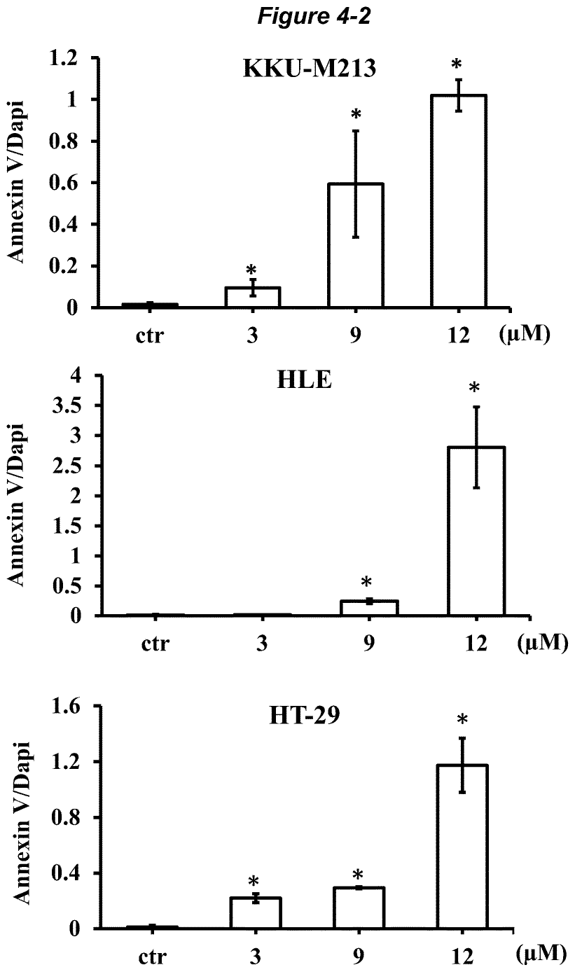

FIG. 4 shows Compound 1 induces apoptosis, in a dose dependent manner. Fluorescence microscopy of cells treated with 3, 9 and 12 μM Compound 1 for 4 h and stained with Annexin V-FITC antibody. Annexin V positive cells are early apoptotic cells. Green fluorescence (Annexin V) was quantified using ImageJ software and correlated to DAPI blue fluorescence (nuclei).

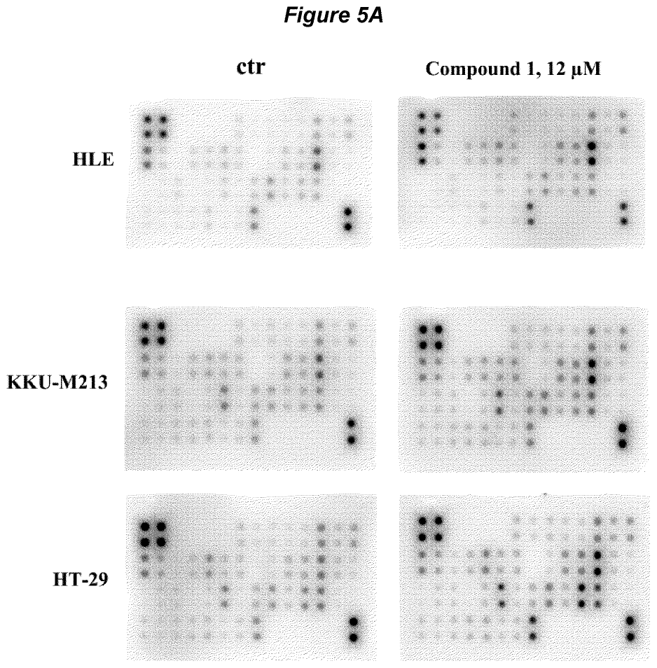

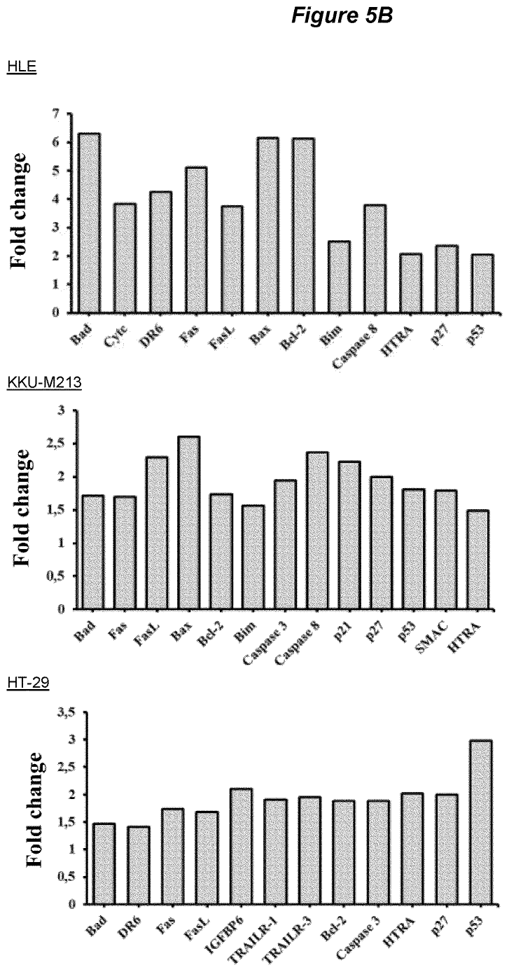

FIG. 5 shows Compound 1 regulates expression of cell apoptosis-related proteins. FIG. 5A shows a list of proteins detected by the human apoptosis antibody array. FIG. 5B shows representative pictures and quantification of the differentially expressed apoptosis-related proteins between untreated control cells and cells incubated with 12 μM Compound 1 for 24 h. The representative images were randomly selected from at least 3 independent experiments.

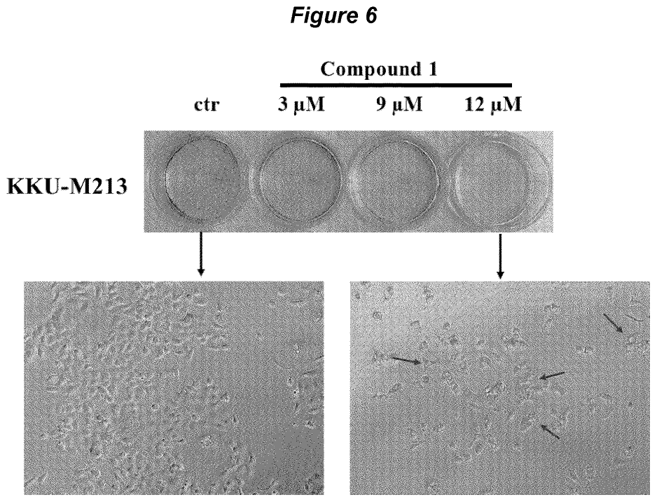

FIG. 6 shows Crystal violet staining (top) and morphological changes (bottom) of KKU-M213 cells observed under an inverted light microscope following treatment with 3, 9 and 12 μM Compound 1 for 72 h: cells exhibit morphological changes and characteristics of apoptosis such as cell shrinkage and cytoplasmic vacuolization (red arrow). *P<0.05

### Effect of Compound 1 on Cell Apoptosis

To further explore the activity of Compound 1 on gastrointestinal cancer cells, the effectiveness on cell apoptosis was investigated. Following incubation with Compound 1, cells underwent morphological changes of cell shrinkage and cytoplasmic vacuolization (FIG. 1C). An early event in apoptosis is represented by the loss of plasma membrane asymmetry, resulting in the translocation of phosphatidylserine (PS) from the inner to the outer plasma membrane leaflet. Exposed PS on the cell surface can be used to measure apoptosis by the binding with Annexin V. KKU-M213, HLE and HT-29 cells were incubated with increasing concentrations of Compound 1 (3, 9 and 12 μM). After 4 hours, cells were stained with a FITC-Annexin V antibody and fixed. In KKU-m213 and HT-29, Compound 1 significantly (p<0.05) induced cell apoptosis at 3, 9 and 12 mM in a dose dependent manner, whereas in HLE cells the same effect was seen at 9 and 12 mM (FIG. 4). Moreover, a human apoptosis antibody array was used to identify the differential expression of 43 apoptosis-related proteins in cells treated with 12 μM Compound 1 for 24 h compared to untreated cells. As shown in FIG. 5, Compound 1 may promote cell apoptosis through upregulating pro-apoptosic proteins including Bim, Caspase-3 and -8, HTRA, DR6, Fas, p53 and SMAC.

### Example 4

### Effect of Compound 1 on Organoid Formation

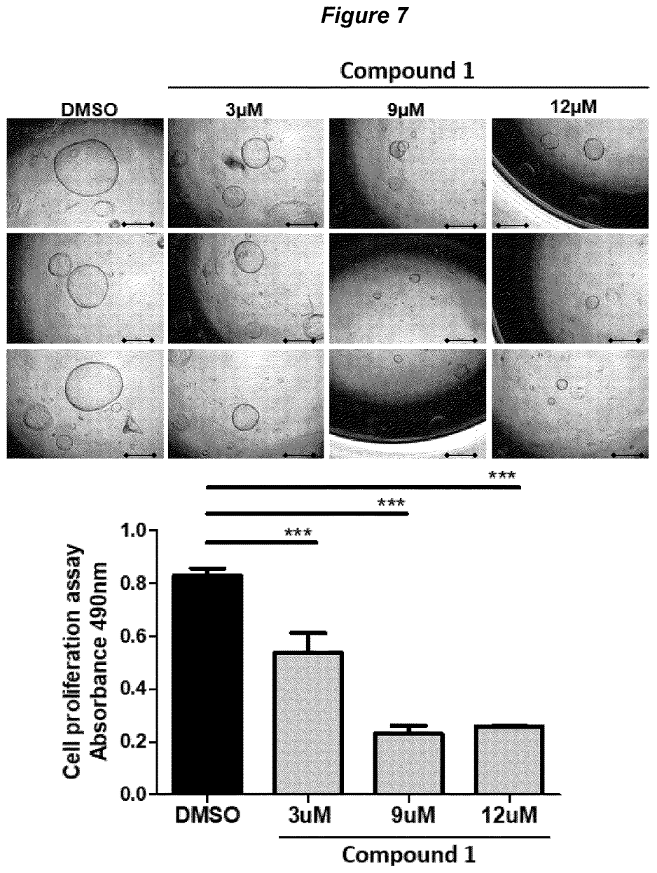

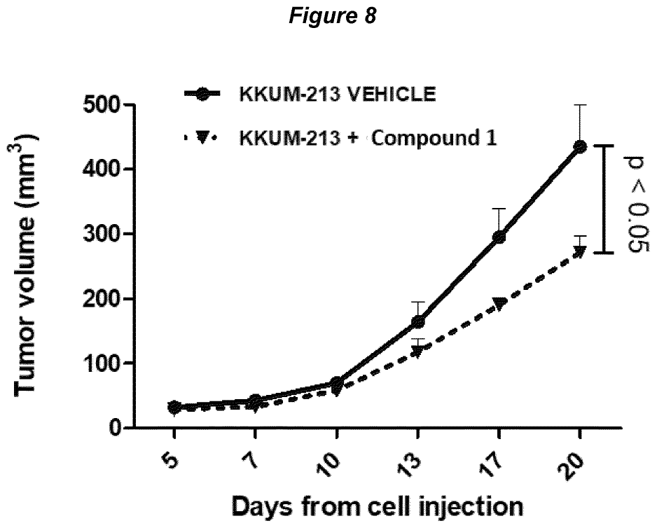

To further explore the activity of Compound 1 on gastrointestinal cancer cells, the effectiveness on organoid formation was investigated. Intrahepatic cholangiocarcinoma cancer cells were isolated from tumour tissues, counted and embedded in 96-well plate with Matrigel. After 3D-model formation, three different concentrations of Compound 1 (3, 9 and 12 μm) were tested in these patient-derived organoid (PDO) cultures. As shown in FIG. 7, bright-field images and cell proliferation assay measurements show significant organoid growth inhibition after 14 days of Compound 1 treatment compared to vehicle. Scale bars correspond to 50 μm, Data are presented as the mean±SD; ***P<0.001.

### Example 5

### Effect of Compound 1 on Tumour Growth In Vivo

To further explore the activity of Compound 1 on gastrointestinal cancer cells in vivo, the effectiveness in a mouse model of cholangiocarcinoma was investigated. Xenografted tumours were produced by co-injection of KKUM-213 cells and human cancer-associated fibroblasts (CAFs). Starting from the 5th day the mice were treated twice daily with Compound 1 (10 mg/kg) or vehicle (1% (w/v) methylcellulose in water) by oral gavage. The tumour volumes were monitored twice a week until the animals were sacrificed. As shown in FIG. 7 tumour volume measurements demonstrate that Compound 1 significantly reduces tumour progression compared to the vehicle group. Data are presented as the mean±SD (n=5 mice per group; Mann-Whitney test:*P<0.05).

### REFERENCES

A number of publications are cited above in order to more fully describe and disclose the invention and the state of the art to which the invention pertains. Full citations for these references are provided below. The entirety of each of these references is incorporated herein:
WO2016/124939
Arnold et al Gastroenterology 159(1) July 2020 10.1053/j.gastro.2020.02.068

For standard molecular biology techniques, see Green, M. and Sambrook, J., Molecular Cloning, A Laboratory Manual. 4th ed. 2012, Cold Spring Harbor, New York: Cold Spring Harbor Laboratory Press

1-8. (canceled)

9. A method of treating a non-pancreatic gastrointestinal cancer in a subject in need thereof, comprising administering to the subject a compound of formula I: 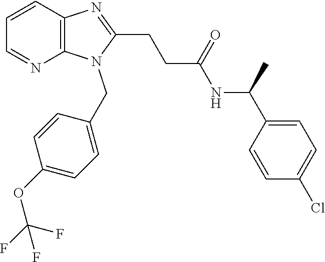 [SMILES: C[C@H](NC(=O)CCc1nc2cccnc2n1Cc1ccc(OC(F)(F)F)cc1)c1ccc(Cl)cc1]
or a pharmaceutically acceptable salt thereof.

10. The method according to claim 9, wherein the non-pancreatic gastrointestinal cancer is selected from liver cancer, cholangiocarcinoma or colorectal cancer.

11. The method according to claim 9, wherein the non-pancreatic gastrointestinal cancer is liver cancer.

12. The method according to claim 9, wherein the non-pancreatic gastrointestinal cancer is cholangiocarcinoma.

13. The method according to claim 9, wherein the non-pancreatic gastrointestinal cancer is colorectal cancer.

14. The method according to claim 9, wherein the compound of Formula I is administered in a pharmaceutical composition comprising said compound or a pharmaceutically acceptable salt thereof and a pharmaceutically acceptable diluent, carrier or excipient; wherein the pharmaceutical composition is suitable for oral administration.

15. The method according to claim 9, wherein the method comprises administration of a therapeutically effective amount of an additional chemotherapeutic agent.

16. The method according to claim 15, wherein the additional chemotherapeutic agent is selected from gemcitabine, cisplatin, doxorubicin, mitoxantrone, 5-fluorouracil (5-FU), capecitabine, irinotecan, oxaliplatin, trifluridine and tipiracil

17-24. (canceled)

25. The method according to claim 11, wherein the compound of Formula I is administered in a pharmaceutical composition comprising said compound or a pharmaceutically acceptable salt thereof and a pharmaceutically acceptable diluent, carrier or excipient; wherein the pharmaceutical composition is suitable for oral administration.

26. The method according to claim 11, wherein the method comprises administration of a therapeutically effective amount of an additional chemotherapeutic agent.

27. The method according to claim 26, wherein the additional chemotherapeutic agent is selected from gemcitabine, cisplatin, doxorubicin, mitoxantrone, 5-fluorouracil (5-FU), capecitabine, irinotecan, oxaliplatin, trifluridine and tipiracil.

28. The method according to claim 12, wherein the compound of Formula I is administered in a pharmaceutical composition comprising said compound or a pharmaceutically acceptable salt thereof and a pharmaceutically acceptable diluent, carrier or excipient; wherein the pharmaceutical composition is suitable for oral administration.

29. The method according to claim 12, wherein the method comprises administration of a therapeutically effective amount of an additional chemotherapeutic agent.

30. The method according to claim 9 wherein the additional chemotherapeutic agent is selected from gemcitabine, cisplatin, doxorubicin, mitoxantrone, 5-fluorouracil (5-FU), capecitabine, irinotecan, oxaliplatin, trifluridine and tipiracil.

31. The method according to claim 13, wherein the compound of Formula I is administered in a pharmaceutical composition comprising said compound or a pharmaceutically acceptable salt thereof and a pharmaceutically acceptable diluent, carrier or excipient; wherein the pharmaceutical composition is suitable for oral administration.

32. The method according to claim 13, wherein the method comprises administration of a therapeutically effective amount of an additional chemotherapeutic agent.

33. The method according to claim 32, wherein the additional chemotherapeutic agent is selected from gemcitabine, cisplatin, doxorubicin, mitoxantrone, 5-fluorouracil (5-FU), capecitabine, irinotecan, oxaliplatin, trifluridine and tipiracil.
